(19) United States
(12) Patent Application Publication
(10) Pub. No.: US 2004/0082669 A1
(43) Pub. Date: Apr. 29, 2004

(54) Operating conditions for copper-based water-gas shift catalysts

(76) Inventors: Wolfgang Friedrich Ruettinger, East Windsor, NJ (US); Oleg Mikhailovich Ilinich, Monmouth Junction, NJ (US); Robert J. Farrauto, Princeton, NJ (US)

Correspondence Address: Chief Patent Counsel, Engelhard Corporation, 101 Wood Avenue, P.O. Box 770, Iselin, NJ, 08830-0770, US

(21) Appl. No.: 10/664,286

(22) Filed: Sep. 17, 2003

Related U.S. Application Data
(63) Continuation-in-part of application No. 10/281,635, filed on Oct. 28, 2002.

(51) Int. Cl. C07C 27/06

(52) U.S. Cl. 518/703

(57) ABSTRACT

Provided is a method for operating a water-gas shift reactor that extends the useful operating life of copper-based catalysts contained therein. Methods of the invention are especially useful, for example, in operating water-gas shift reactors that are subject to frequent cycles of startups and shutdowns.

In another aspect, the invention relates to a copper-based catalyst, which is useful, among other thing for catalyzing the water-gas shift reaction. The catalyst contains from 5 to 20 wt. % of a copper component, wherein at least 50 wt. % of the copper component is in the form of a copper oxide, aluminum oxide spinel; at least 75 wt. % of alumina; and from 0.03 to 1 wt. % of carbonaceous residue.

This application is a continuation-in-part of U.S. patent application Ser. No. 10/281,635, filed Oct. 28, 2002, the disclosure of which is hereby incorporated by reference as if fully set forth herein.

The present invention relates to methods for operating copper-based water gas shift catalysts that extend their useful catalytic lifetime. Such methods are particularly useful in operating water-gas shift reactors that are subject to frequent startup and shutdown cycles. The methods of the invention are useful, for example, in generating hydrogen in the gas stream supplied to fuel cells, which are often operated with frequent cycles of startups and shutdowns.

Fuel cells require both oxygen and a source of hydrogen to function. The oxygen can be readily obtained in pure form (i.e., as O₂) or from the air. The low volumetric energy density of isolated hydrogen gas compared to conventional hydrocarbon fuels however, makes the direct supply of hydrogen gas to fuel cells impractical for most applications. Large volumes of hydrogen gas are needed to provide the equivalent amounts of energy that are stored in much smaller volumes of conventional hydrocarbon fuels such as natural gas, alcohol, oil or gasoline. Accordingly, the conversion of known hydrocarbon-based fuel stocks to hydrogen gas is an attractive source of hydrogen for fuel cells and other applications.

Hydrocarbon-based fuel stocks are converted to hydrogen and carbon dioxide by a series of conversions that typically include steam reforming and/or partial oxidation, and water-gas shift reactions. These conversions can be conducted in a fuel cell processor (also known as a fuel cell steam reformer).

The conversions that occur within the fuel cell processor not only produce a hydrogen stream but also lower the concentration of carbon monoxide in the hydrogen stream to levels that are acceptable for delivery to the fuel cell processor. Achieving a sufficiently low level of carbon monoxide in the hydrogen stream that is ultimately supplied to the fuel cell is important to minimize carbon monoxide-induced degradation of platinum-based electrodes in the fuel cells. The platinum electrodes are susceptible to even very low levels of carbon monoxide. Water-gas shift conversions are typically well-suited for handling the carbon monoxide concentrations of about 6-12% by volume (on a dry basis) of carbon monoxide that emerges from the steam reforming conversions. The water-gas shift reaction is a well known reaction which is used, among other things, to generate hydrogen in a gas-borne stream by chemical reaction of carbon monoxide with water vapor (H₂O) according to the following stoichiometry:

CO+H₂O→CO₂+H₂

The reaction generally requires a catalyst.

In fuel cell applications, water-gas shift conversions are often conducted in two stages that are fluidly connected: a high temperature stage that is conducted at temperatures of about 350-500° C., and a low temperature stage that is typically conducted at temperatures below about 300° C. Higher temperatures are favorable for water-gas shift processes in terms of higher reaction rates; however, CO conversion in the high temperature process is limited by thermodynamic considerations. Typical catalysts employed in the high temperature stage are iron-chromium-based catalysts. Lower levels of carbon monoxide can be achieved in the low temperature shift stage than in the high temperature stage due to a more favorable thermodynamic equilibrium. Catalysts utilized in the low temperature stage of the water-gas shift reaction are typically copper-based catalysts, such as copper-zinc-alumina compositions.

A problem that plagues many copper-based catalysts is the gradual decline in catalytic activity in the process environment. While in some cases, the decline involves contamination of the catalysts by foreign substances (e.g., sulfur compounds, halides etc.), deactivation of copper-based catalysts may also be associated with exposure to steam. In particular, exposure of copper-based catalysts to steam in the absence of a reducing agent such as carbon monoxide and hydrogen significantly degrades the WGS catalytic performance over time. Exposure of the catalysts under such conditions is most frequently encountered upon startup and shutdown of water-gas shift reactors. For instance, in starting up fuel cell processors, steam reformer reactors are commonly flushed with steam to flush out residual gases, heat up the steam reforming catalyst and prevent coke formation. This practice can expose copper-based WGS catalysts contained in WGS reactors that are downstream of the steam reformers.

Moreover, in many applications such as some fuel cell applications, WGS reactors are operated with frequent cycles of startups and shutdowns in response to transient power demands. Typically, WGS reactors are started up and shutdown using the process gas, containing steam and carbon monoxide. By operating the WGS reactors in such a manner, it has been found that the catalytic activity declines in a cumulative fashion, where with each cycle of operation, the outlet carbon monoxide concentration rises. Under some operating conditions, the decline in catalytic activity may not become evident until several cycles of operation have been conducted.

Deactivated copper-based catalysts are not easily regenerated in most water-gas shift applications. Moreover, frequent recharging of reactors with fresh catalysts is impractical in many water-gas shift applications, including most fuel cell applications. Methods for operating low temperature water-gas shift catalysts that extend the useful operational lifetime of copper-based catalysts in applications that involve frequent cycles with startups and shutdowns are therefore desirable.

### SUMMARY OF THE INVENTION

In one aspect, the invention relates to a multi-cyclic method of operating a water-gas shift (WGS) reactor having a copper-based WGS catalyst. The method includes:

(a) heating the WGS catalyst in its oxidized state in a substantially dry, reducing gas containing a reducing component to a temperature of 100 to 350° C. to form a reduced WGS catalyst;

(b) flowing a reactant gas comprising CO and H₂O through the reactor to contact the reduced WGS catalyst, while maintaining the WGS catalyst temperature at an operating temperature of 150 to 350° C.;

(c) purging the WGS reactor with a substantially dry, first non-oxidizing gas at the operating temperature, and allowing the WGS catalyst temperature to fall below the operating temperature;

(d) heating the WGS catalyst to the operating temperature in a substantially dry, second non-oxidizing gas; and

(e) repeating (b) through (d) at least two times.

In one embodiment of the multi-cyclic method, the reducing gas of (a) contains at least 1% by volume of CO, H₂, or a mixture thereof. Such compositions can be obtained, for example, from an upstream reformer reactor operating in catalytic partial oxidation mode.

The substantially dry, first and second non-oxidizing gases typically contain at least one of nitrogen, argon, de-sulfured gaseous hydrocarbon, carbon dioxide, carbon monoxide and hydrogen. In some embodiments, the substantially dry, first and second non-oxidizing gases can have the same composition.

In some embodiments of the multi-cyclic method, the reactant gas of (b) also contains at least 10% by volume H₂, on a dry basis in addition to CO and H₂O.

In a preferred embodiment of the multi-cyclic method, the copper-based, WGS catalyst contains from 5 to 20 wt. % of a copper component, wherein at least 50 wt. % of the copper component is in the form of a copper oxide, aluminum oxide spinel; and at least 75 wt. % of alumina. In this preferred method, the WGS catalyst in its oxidized state is preferably heated in the reducing gas at a temperature below 300° C. to form the reduced WGS catalyst in (a). Also in this preferred method, the reduced WGS catalyst is preferably contacted with the reactant gas, while maintaining the WGS catalyst temperature at an operating temperature of 180 to 300° C. in (b).

Another aspect of the invention relates to a copper-based catalyst, which is useful, among other things for catalyzing the water-gas shift reaction. The catalyst contains from 5 to 20 wt. % of a copper component, wherein at least 50 wt. % of the copper component is in the form of a copper oxide, aluminum oxide spinel; at least 75 wt. % of alumina; and from 0.03 to 1 wt. % of carbonaceous residue.

In another aspect, the invention relates to a method of conducting the water-gas shift reaction that includes contacting a reactant gas stream comprising CO and H₂O with a WGS catalyst to form H₂ and CO₂. The WGS catalyst contains from 5 to 20 wt. % of a copper component, wherein at least 50 wt. % of the copper component is in the form of a copper oxide, aluminum oxide spinel; at least 75 wt. % of alumina; and from 0.03 to 1 wt. % of carbonaceous residue.

### BRIEF DESCRIPTION OF THE DRAWINGS

FIG. 1 shows a graph of a temperature vs. time program that models a single catalyst operational cycle used in conducting the catalyst deactivation studies in Example 2.

FIG. 2 shows the outlet CO concentration as a function of time for five catalyst trials conducted under varying conditions in the startup and shutdown stages of the operational cycle.

FIG. 3 shows a comparison of the catalyst aging rate (X) for the five different catalyst trials of Example 2.

FIG. 4 shows a comparison of the aging rates for two copper-based, WGS catalysts.

### DEFINITIONS

The following terms shall have, for the purposes of this application, the respective meanings set forth below.

“Copper-Based Catalyst” refers to a catalyst containing at least 5 wt. % of a copper component, and which requires a copper component to have catalytic activity in the desired operating condition.

“Copper component,” as used herein, means copper (metal) and/or oxides thereof.

“Non-Oxidizing Gas” refers to gas streams containing less than 0.1% by volume of O₂, or other gaseous oxidant. In addition, such gases contain less than 0.001% of sulfide components (e.g., H₂S, mercaptans) that would react substantially with the copper catalytic surface.

“Reducing Gas” refers to a non-oxidizing gas that contains a reducing component, and preferably, at least 1% by volume of H₂, CO or a mixture thereof.

“Substantially dry” when used to describe gas streams, refers to gas streams having less than 5% by volume of H₂O.

“Substantially no O₂” when used to describe gas streams, refers to gas streams having less than 0.1% by volume of O₂.

### DETAILED DESCRIPTION OF THE INVENTION

Provided is a method for operating a water-gas shift reactor that extends the useful operating life of copper-based catalysts contained therein. Methods of the invention are especially useful, for example, in operating water-gas shift reactors that are subject to frequent cycles of startups and shutdowns.

Applicants have identified certain operational conditions relating to the gas composition, the gas temperature and that catalyst oxidation state that are deleterious to the useful catalyst life of copper-based WGS catalysts. In one aspect, Applicants have found that as a result of reducing an oxidized, copper-based WGS catalyst in the presence of steam, the so-formed reduced catalyst is vulnerable to enhanced catalyst deactivation due to steam exposure. This vulnerability is reflected in a rapid rate of catalyst deactivation after several cycles of reactor startups and shutdowns. Such conditions prevail, for instance, in reducing copper-based WGS catalysts with wet (i.e., steam-containing) reformate gas, a practice that is commonly performed.

In another aspect, Applicants have found that exposure of the copper-based catalyst compositions to a wet gas composition in the absence of a reducing component (e.g. H₂, CO) is deleterious to the catalyst lifetime at any temperature. As described above, such situations are commonly encountered in applications for hydrogen generation where upstream steam reformers are purged with high steam-containing gases at startup, causing the downstream WGS catalysts to be exposed to steam in the absence of a reducing agent.

In a related aspect, exposure of copper-based WGS catalysts below the operating temperature to steam-containing compositions, even with a reducing component present, also accounts for a rapid rate of catalyst deactivation.

Accordingly, Applicants describe herein a multi-cyclic method of operating a water-gas shift (WGS) reactor that avoids the rapid catalyst deactivation due to exposure of copper-based catalysts to the above-described conditions. By the term “multi-cyclic” it is meant that the WGS reactor is operated with at least two cycles, and more preferably, with at least three cycles. The method includes:

(a) heating the WGS catalyst in its oxidized state in a substantially dry, reducing gas comprising a reducing component to a temperature of 100 to 350° C. to form a reduced WGS catalyst;

(b) flowing a reactant gas comprising CO and H₂O through the reactor to contact the reduced WGS catalyst, while maintaining the WGS catalyst temperature at an operating temperature of 150 to 350° C.;

(c) purging the WGS reactor with a substantially dry, first non-oxidizing gas at the operating temperature, and allowing the WGS catalyst temperature to fall below the operating temperature;

(d) heating the WGS catalyst to the operating temperature in a substantially dry, second non-oxidizing gas; and

(e) repeating (b) through (d) at least two times.

Each cycle has: (1) an operating stage that converts CO and H₂O into H₂ and CO₂ at an operating temperature of 150 to 350° C.; (2) a shutdown stage where the temperature of the catalyst is allowed to fall below the operating temperature in the presence of a substantially dry, non-oxidizing gas; and (3) a startup stage where the WGS catalyst is heated to the operating temperature in a substantially dry, non-oxidizing gas. The substantially dry, non-oxidizing gas used in the shutdown and startup stages may be the same composition, or a different composition.

The multi-cyclic method also includes a reducing stage that is conducted on an oxidized, copper-based WGS catalyst to form a reduced WGS catalyst. The reducing stage includes heating the WGS catalyst in a substantially dry, reducing gas at a temperature below 350° C., e.g. from 100 to 350° C., and preferably from 150 to 350° C. The reducing gas used in the method is a substantially dry, non-oxidizing gas that contains a reducing component, and that preferably contains at least 1% by volume of a reducing component selected from CO, H₂ or a mixture thereof. Other components that can also be included in the reducing gas include conventional inert gases such as nitrogen and argon, de-sulfured gaseous hydrocarbons (e.g., de-sulfured LPG, de-sulfured natural gas), carbon dioxide, and mixtures thereof. For example, the outlet stream from a catalytic partial oxidation can be a suitable reducing gas which contains both hydrogen and carbon monoxide, as well as CO₂. It has been found that by reducing the oxidized catalyst in a substantially dry gas stream, the reduced catalyst that results achieves the highest possible catalytic activity in the WGS reaction. Moreover, after reduction in a substantially dry gas stream, the catalyst shows a much more stable activity over extended operating periods than the same catalyst that is reduced in a steam-containing reducing gas.

The term “operating temperature” refers to the temperature range where the copper-based, WGS catalyst has activity in catalyzing the reaction of CO with H₂O to form H₂ and CO₂. Above the upper end of this temperature range, the catalyst begins to lose activity, and is thereby rendered less catalytically active, possibly due to the loss of active, catalytic surface area. Operating temperatures for copper-based catalysts can vary depending on the compositions of the catalysts, as well as on some of the other operating parameters such as space velocity. Preferred operating temperatures for copper-based water-gas shift catalysts can be determined by any method known to those of skill in the art such as by monitoring gas compositions exiting the WGS reactor along with the catalyst temperature. Typically, copper-based WGS catalysts have an operating temperature between 150 and 350° C.

By practicing the inventive method, the cumulative decline in catalytic activity that is typically associated with prolonged use of copper-based catalysts that are operated with multiples cycles of startups and shutdowns, is significantly decreased or prevented. Preferably, the water-gas shift activity of the catalyst retains at least 50% of the initial catalyst activity after five cycles.

In accordance with the invention, the copper-based catalyst is maintained in a reduced state after the reduction stage has been conducted. The catalyst retains high catalytic activity by avoiding exposure of the catalyst to wet gas streams in the absence of a reducing agent at any temperature. In addition, exposure to wet gas streams in the presence of a reducing component should be avoided below the operating temperature. Therefore, during operations where the temperature of the catalyst falls below the operating temperature, such as during the startup and shutdown stages, the catalyst is only exposed to substantially dry and non-oxidizing gas streams to avoid any deleterious effects on the catalytic activity.

Among other things, the startup stage involves bringing the catalyst up to its operating temperature in the presence of a substantially dry, non-oxidizing gas to avoid exposure of the catalyst to a wet gas stream at temperatures below about 150° C. Suitable substantially dry, non-oxidizing gases for conducting the startup stage include conventional inert gases such as nitrogen and argon, de-sulfured gaseous hydrocarbons (e.g., de-sulfured LPG, de-sulfured natural gas), carbon dioxide, and mixtures thereof. Since the WGS catalyst is now in its reduced state, it is unnecessary to include a reducing component in the gas stream used in the startup stage; however, substantially dry, reducing gases such as carbon monoxide, hydrogen, and mixtures thereof are optionally included in the gas contacting the catalyst.

In hydrogen generation for fuel cells and other applications, the catalyst can be brought to its operating temperature by, for example, exposure to a gas stream that is at an elevated temperature. For example, heated gas streams containing substantially dry, non-oxidizing gases can be used. Here again, outlet streams from catalytic partial oxidation reactors can also be used. There may also be provisions for supplying heat to the WGS reactor such as heated electrical coils, steam jackets or other heat exchange provisions.

Once the copper-based WGS catalyst has been heated to the operating temperature, the catalyst can be exposed to a reactant gas containing CO and H₂O so that hydrogen can be generated. The reactant gas can be, for example, a reformate stream from a steam reformer reactor or autothermal reformer reactor. The composition of the reactant gas can vary depending on the source of the gas. Although higher proportions of carbon monoxide can be accommodated in the reactant gas, the method is particularly effective where the carbon monoxide concentration is less than about 15% by volume, on a dry basis. Typically, molar excesses of steam are used relative to the amount of carbon monoxide present in the input gas stream. Generally, H₂O:CO molar ratios of between 1:1 (i.e., “1.0”) and 20:1 (i.e. “20.0”) are preferred in the reactant gas, with higher ratios being particularly preferred for high conversion of carbon monoxide.

In hydrogen generation for fuel cells and other applications, reactant gas streams used in the operating stage typically contain at least 10% by volume of hydrogen on a dry basis in addition to the carbon monoxide and steam. Higher concentrations, e.g., greater than 30-40% by volume, are often utilized in hydrogen generation for fuel cells and other applications. In addition to carbon monoxide, steam and hydrogen, the reactant gas can contain carbon dioxide, nitrogen, and minor amounts of hydrocarbons (e.g., paraffins, olefins), alcohols and aldehydes. Preferably, the reactant gas contains about 4-5% or less by volume of hydrocarbons (or alcohols, aldehydes) and about 25% or less by volume of carbon dioxide on a dry basis.

The temperature of the WGS catalyst is typically maintained between 150 and 350° C. during the operating stage. Reaction zone pressure is preferably maintained below the dew point pressure of the reactant gas. It should be recognized that lower or higher reaction zone pressures can be used, such as from atmospheric up to about 500-700 psig.

For reactors operated with particulate WGS catalysts, gaseous hourly space velocities of about 10 to about 10,000 hr⁻¹ VHSV measured on the basis of dry gas under standard conditions are suitable for most operations such as hydrogen generation for fuel cells and other applications. Preferred gaseous hourly space velocities are from 500 to 3000 hr⁻¹ VHSV.

When the demand for hydrogen generation has ceased, the WGS reactor is shut down by purging the reactor with a substantially dry, non-oxidizing gas at the operating temperature, and then allowing the WGS catalyst temperature fall below the operating temperature. Suitable substantially dry, non-oxidizing gases for purging the WGS reactor include conventional inert gases such as nitrogen and argon, de-sulfured gaseous hydrocarbons (e.g., de-sulfured LPG, de-sulfured natural gas), carbon dioxide, and mixtures thereof. As will be apparent to those of skill in the art oxygen (as well as other oxidants) and sulfide components (e.g., H₂S, mercaptans) that may oxidize, or would otherwise react substantially with the copper catalytic surface should be avoided.

The exclusion of water during the shutdown stage is crucial to maintaining the activity of the copper-based catalysts over prolonged periods of operation. Exposure of the catalysts to water during the shutdown stage is particularly damaging to their activity in the absence of a reducing component such as CO or H₂.

Preferred Copper-Based Compositions

A preferred copper-based WGS catalyst for conducting the inventive method is a coextruded mixture of a copper component and alumina. Preferably at least 50% of the copper component is in the form of a copper oxide, aluminum oxide spinel. These preferred copper-based WGS catalysts have an operating temperature of about 150 to 350° C. It has been found that these catalysts are substantially less self-heating upon exposure to air than conventional, copper-based WGS catalysts, such as copper-zinc WGS catalysts. This property makes the catalysts desirable, for example, for implementation in fuel cell processors where minimizing the risk of an exothermic temperature rise is of particular concern.

The catalysts generally contain about 5-20 wt. %, and more preferably 10-20 wt. % of a copper component. Lower levels of copper component than those specified can result in a composition that has insufficient WGS catalytic activity. Higher levels of copper component than those specified are less preferred due to the increased self-heating liability associated with higher copper loadings.

At least some, and preferably at least 50%, of the copper component in the composition is in the form of the copper oxide, aluminum oxide spinel. As will be readily apparent to those of skill in the art, copper oxide, aluminum oxide spinel has the general formula CuAl₂O₄. Compositions containing such spinel structures can be readily detected using well-known spectroscopic techniques such as by determination of X-ray diffraction patterns. While not being bound by any specific theory, it is believed that the spinel structure in the catalyst enhances the catalyst's carbon monoxide conversion activity, and also contributes to the improved durability of the catalysts in high steam, low-temperature environments relative to copper-based catalysts where the spinel structure is absent.

Preferably, the composition contains at least 75 wt. % of alumina, and more preferably at least 80 wt. %. Higher proportions of alumina provide the catalysts with a higher overall heat capacity to contribute to the reduced exothermic liability that characterizes the catalysts of the invention. Alumina has a higher specific heat capacity than many other common components of catalyst compositions. The alumina utilized in the invention is preferably activated alumina. As used herein, the terms “activated alumina” refer to a high BET surface area alumina, for example, an alumina having a surface area greater than 10 m²/g, preferably greater than 60 m²/g. The activated alumina is composed of one or more of gamma, theta and delta aluminas.

Other catalyst additives can be included in the composition such as promoters and stabilizers. These additional catalyst additives are typically present in the final catalyst composition (i.e., in the calcined catalyst composition) in less than 10 wt. %, preferably less than 5 wt. %, and more preferably less than 2 wt. % to ensure that the catalyst has a sufficient proportion of alumina to abate any temperature rises due to changes in the copper component's oxidation state.

In a preferred embodiment of the invention, the composition contains a carbon precursor. The term “carbon precursor” refers to any additive that is added to the catalyst composition that upon calcination decomposes and volatilizes to leave carbonaceous residue in the composition. Exemplary carbon precursors include wood flour; carbon powder; graphite powder; furfuryl alcohol; monosaccharides and polysaccharides such as starch, sucrose, lactose, maltose, cellulose, cellulose derivatives (e.g., carboxymethycellulose); water-soluble or water-dispersible polymers such as polyethylene glycol, polyethylene oxide, polypropylene oxide, polyvinylalcohol and the like. Preferably, the amount of carbon precursor introduced into the composition is from 1 to 20 wt. % of the total catalyst precursor composition, where the catalyst precursor composition contains copper precursor, carbon precursor and insoluble alumina precursor (as described below). Upon calcination the amount of carbonaceous residue resulting in the calcined composition is typically 0.03 to 1 wt. %.

Typically, catalyst additives such as wood flour have been added to improve the porosity of calcined catalyst compositions. For the preferred copper-based compositions described herein, improved porosity was not observed. Surprisingly however, a significant increase in the useful catalyst lifetime was observed for a copper-based catalyst prepared with the carbon precursor, wood flour, than in an otherwise identical copper-based catalyst prepared without addition of wood flour (see Example 4, below).

Another desirable property associated with the copper-based catalyst prepared with wood flour is that a higher CO conversion activity is observed when the oxidized catalyst is reduced and operated with a high steam-containing reformate gas (e.g., 26 vol. %), compared to an otherwise identical composition that did not include a carbon precursor (see Example 3). This property is advantageous in instances where it may be inconvenient to initially reduce the catalyst composition in a substantially dry, non-oxidizing gas that contains a reducing component.

The catalysts are prepared by a coextrusion procedure. A mixture of a copper precursor, an alumina precursor, a carbon precursor (e.g., wood flour) and an aqueous solvent are combined in a fashion to adequately disperse the copper precursor and the alumina precursor to form a paste. As used herein, the terms “copper precursors” refer to copper compounds such as salts or complexes that are water-soluble or water-dispersible that upon calcination form copper, an oxide of copper or mixtures thereof. Copper precursors can form copper oxide, aluminum oxide spinel in the presence of an alumina precursor upon calcination at temperatures above 600° C. Preferred copper precursors include copper nitrate, -acetate, -carbonate, -formate and -lactate.

The terms “alumina precursor” refer to aluminum compounds that upon calcination form aluminum oxide (also known as “alumina”), and in the presence of a copper precursor also form copper oxide, aluminum oxide spinel. Preferred aluminum precursors are water-insoluble compounds such as hydrated forms of aluminum oxide e.g., boehmite, gibbsite, hydrargillite, bayerite, nordstrandite and diaspore.

The copper precursor is preferably mixed with an insoluble alumina precursor. Conducting the mixing of the copper precursor with the alumina precursor in this fashion, as opposed to using other techniques such as formation of mixed metal oxides, ensures that substantially all of the copper component is supported on alumina particles. This morphology ensures efficient heat transfer from copper components on to the alumina which has a higher specific heat capacity than does the copper component. In addition, the composition's morphology prevents unwanted sintering of copper particles which can occur if the copper particles are inadequately supported.

In contrast to the method described above, precipitating or gelling mixtures of mixed metal oxides formed from soluble alumina and soluble copper precursors often results in copper and alumina particles that are formed simultaneously. As a result, such mixed metal oxides are characterized by a morphology where the alumina and copper particles are of similar size and a significant proportion of the copper component is in the form of discrete particles. As such, this proportion of the copper component is unsupported by the alumina, and therefore, the efficient heat transfer between the copper component and alumina cannot be achieved.

The mixing of the copper precursor and the insoluble alumina precursor can be conducted by any means that achieves uniform dispersion of the copper precursor on to the alumina precursor. In the extrusion step, the paste is pressed through a die of the desired shape and size to form the coextrudate. The coextrudate is then dried, typically at temperatures of about 80-120° C.

The dried coextrudate is then heated to a temperature of at least 600° C. in an oxidizing atmosphere such as air. Calcination temperatures of at least 600° C. ensure that the higher proportions, e.g., at least 50% of the copper component are actually in the form of a copper oxide, aluminum oxide spinel. The calcination is typically conducted in the range of 600 to 900° C., and preferably in the range of about 650 to 800° C. Preferably, the calcined coextrudate has an effective size not exceeding 5 mm, wherein the terms “effective size” means the minimum dimension (i.e., diameter or length, whichever is less) in the coextrudate particle.

While not wishing to be bound by any specific theory, it is believed that both the coextrusion step and the higher temperatures used in the calcination step contribute to the high mechanical strength of the finished catalyst. The high mechanical strength makes the catalyst more resistant to vibrational forces and other wear mechanisms. These steps also contribute to improved resistance to high steam, low temperature environments and reduced liability to self-heating.

The following examples further illustrate the present invention, but of course, should not be construed as in any way limiting its scope.

### EXAMPLE 1

### Preparation of Copper Aluminum Oxide Spinel Catalyst Extrudate Containing Wood Flour

1 kg of alumina monohydrate Al₂O₃×H₂O was mixed with 30 g of wood flour. 1.5 liters of aqueous solution of Cu(NO₃)₂ containing 5.1 wt. % of copper as metal were added to the above mix. Mixing was continued at ambient conditions until a uniform thick paste was obtained. The paste was extruded from an extruder equipped with an appropriate die to form a 1/16 inch diameter extrudate. The extruded material was dried in air at 80-120° C. and calcined in air at 600-850° C. to produce the catalyst, containing 15 wt. % of copper in the composition. This catalyst is designated as Catalyst A1.

### EXAMPLE 2

### Catalyst Deactivation Studies

In this experiment, the effects on the useful catalyst lifetime due to exposure of the copper-based extrudates prepared in Example 1 to various gas compositions during the reduction, startup and shutdown stages were determined.

The trials were conducted in microflow reactors controlled by a microcomputer that were run continuously. In each trial, 50 cc of the catalyst extrudate as prepared in Example 1 was loaded into a 1″ ID tubular microflow reactor. The flow rate of the feed gas was 1.5 standard liters per minute (SLPM).

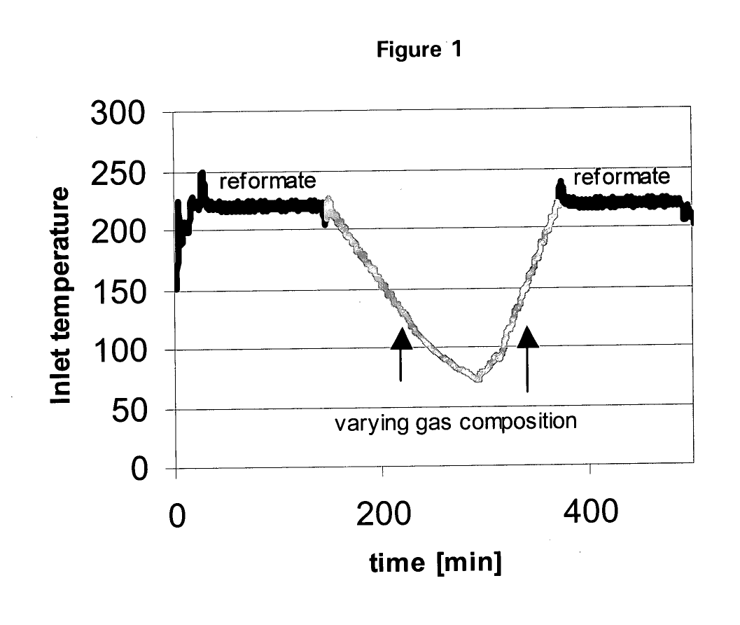

The catalyst samples were subjected to multiple cycles where each cycle had (1) a reactor startup stage; (2) a measurement stage—exposure to wet reformate at 220° C. (or operating stage); and (3) a reactor shutdown stage. The catalyst activity was determined in each cycle by measuring the concentration of CO remaining in the outlet of the microflow reactor during the measurement stage. A single cycle of the temperature vs. time program used in the trials is shown in FIG. 1.

In addition, in each of the trials there was a single catalyst reduction stage conducted on the oxidized catalyst to reduce the copper component of the catalyst, which is not shown in FIG. 1. The reduction stage was conducted in the trials immediately prior to the first measurement stage. In the reduction stage, the catalyst temperature was raised from room temperature to 220° C. with gas streams that contained a reducing component.

The various gas compositions used in each stage of the cycle are listed in Table 1 below. In this experiment, the gas compositions used in the startup and shutdown stages were identical within any given trial, and therefore are identified as “Shutdown/Startup” in Table 1.

TABLE 1

| Description | Gas Composition | Where Used in Trials |
|---|---|---|
| Dry | 7% CO, 48% H₂, 11% CO_{2,} | Reduction in Trial 1 |
| Reformate | 34% N₂ | |
| Wet | 4.6% CO, 34.6% H₂, 8.2% | Measurement Stage |
| Reformate | CO₂, 24.5% N₂, 28% H₂O | in Trials 1-5, Shutdown/ |
| | | Startup in Trial 1, |
| | | Reduction and Shutdown/ |
| | | Startup in Trial 2 |
| Dry H₂/N₂ | 30% H₂ in N₂ | Reduction in Trial 4 |
| Wet H₂/N₂ | 22% H₂, 33% N₂, 45% H₂O | Reduction in Trial 5 |
| H₂O/N₂ | 45% H₂O, 55% N₂ | Shutdown/Startup in |
| | | Trials 4 and 5 |
| H₂/CO₂ | 30% H₂, 70% CO₂ | Reduction in Trial 3 |
| Dry CO₂ | 100% dry CO₂ | Shutdown/Startup in Trial 3 |

During the reactor startup stage, the inlet temperature of the gas was ramped from 70° C. to 220° C. over the course of 60 minutes. The measurement stage was always conducted at 220° C. in the presence of the wet reformate gas (reactant gas) composition. During the shutdown stage, the inlet temperature of the gas was decreased from 220° C. to 70° C. while being exposed to the gas composition identified in the Shutdown/Startup stages in Table 1. Table 2 provides a summary of the conditions for each of five trials that were conducted with the catalyst extrudates of Example 1.

TABLE 2

| | Gas Used in Reduction | Gas Used in | Gas Used in |
|---|---|---|---|
| | Stage | Measurement Stage | Shutdown/Startup |
| Trial # | (Reducing Gas) | (Reactant Gas) | Stages |
| 1 | dry reformate | wet refonnate | wet reformate |
| 2* | wet reformate | wet reformate | wet reformate |
| 3 | H₂/CO₂ | wet reformate | dry CO₂ |
| 4 | H₂/N₂ | wet reformate | H₂O/N₂ |
| 5* | wet H₂/N₂ | wet reformate | H₂O/N₂ |

Cycles were repeated in each trial over the course of at least 50 hours. In some trials, where the catalyst sample did not show substantial deactivation, the experiment was conducted for longer time periods.

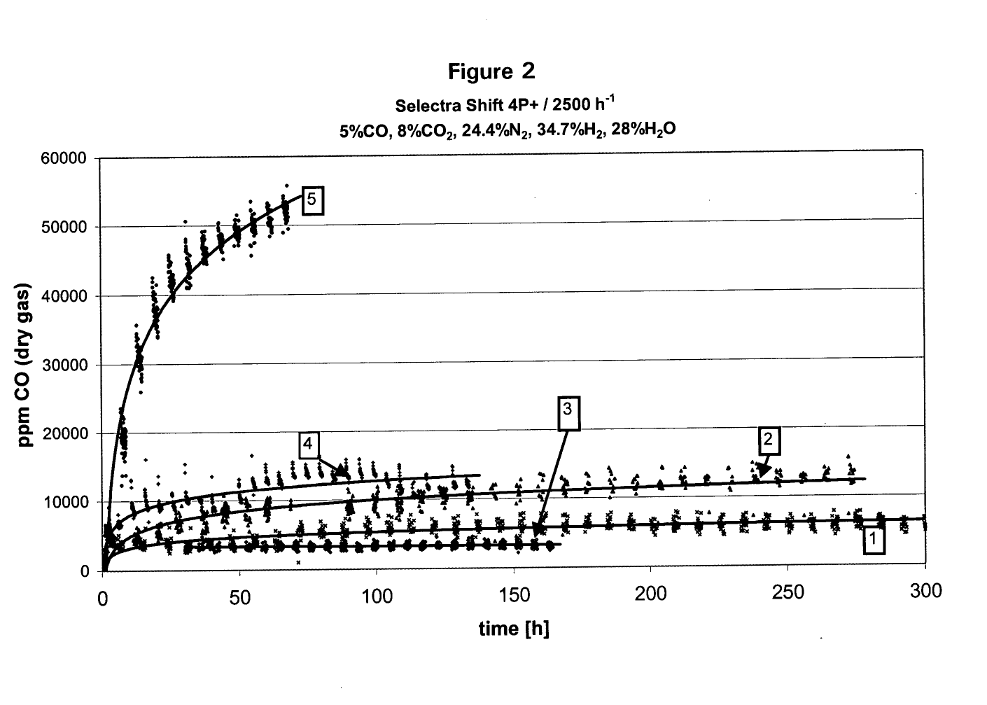

For each cycle, the catalyst activity was characterized by the CO outlet concentration at an inlet temperature of 220° C. measured with an on-line infrared gas analyzer ZRH (Fuji Electric, Japan). The results of the experiment are graphically represented in FIG. 2.

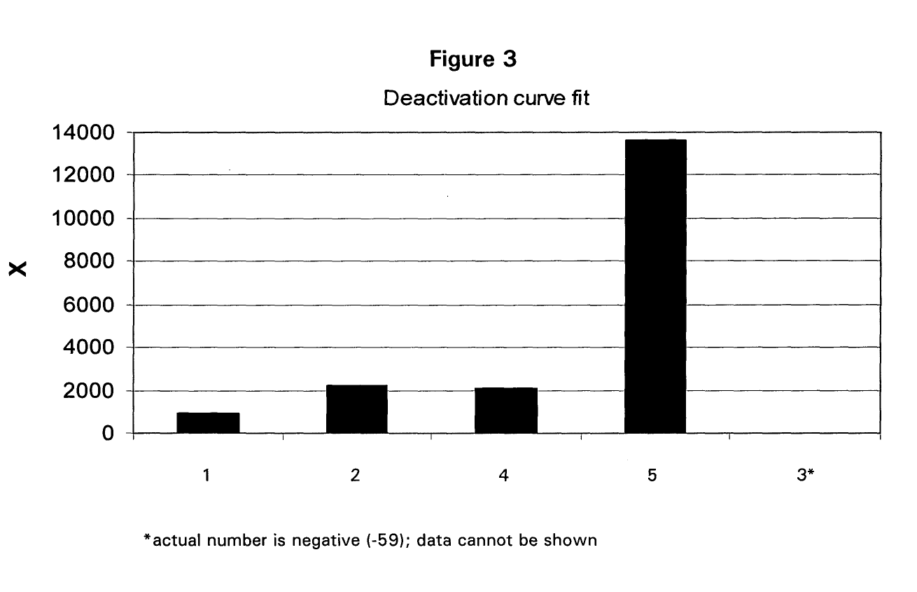

The data curves shown in FIG. 2 can be fit reasonably well with a logarithmic curve of the format

[outlet CO]=X*ln(t)+A

wherein t is the time in hours, and A is a measure of the initial catalytic activity. The parameter X provides a measure of the aging rate for each of the trials. FIG. 3 provides a comparison of the parameter X for each of the trials. As can be seen in the figure, there is no deactivation when the reduction, startup and shutdown stages are conducted in the absence of water (in Trial 3), where dry CO₂ is used as both a diluent in the reduction gas composition and in essentially pure form in the gases used in the startup and shutdown stages. A similar effect has been observed when dry de-sulfured natural gas was used in place of dry CO₂ (data not shown).

FIGS. 2 and 3 also show that exposure of the catalyst to a wet gas composition during the reduction stage coupled with exposure of the catalyst to a wet gas composition in the absence of a reducing agent (i.e., during the startup and shutdown stages) causes the most degradation of the catalyst relative to the other conditions tested.

### EXAMPLE 3

### Evaluation of Water-Gas Shift Activity—Effect of Wood Flour Addition

In this experiment, improvements in the water-gas shift catalytic activity obtained by including the carbon precursor, wood flour in the catalyst composition are demonstrated in a high steam environment. The water-gas shift activity was determined for a catalyst extrudate prepared using wood flour, Catalyst A1 (prepared as in Example 1), and a reference catalyst prepared without wood flour, Catalyst B1. Catalyst B1 was prepared by the same procedure as Catalyst A1, but for the inclusion of wood flour. Catalyst B1 contained the same wt. % of copper as Catalyst A1.

Activity measurements were conducted in a microflow reactor according to the following procedure:

4 g of the catalyst sample was loaded into a 1″ ID microflow reactor and heated up to 90° C. in a dry nitrogen flow at a flow rate of 0.5 SLPM. At 90° C., the nitrogen flow was replaced by a feed gas mixture containing 8% CO, 10% CO₂, 43% H₂, and 39% N₂ (on a dry basis), with steam added in the amount sufficient to yield a feed gas composition containing 26% of steam. This flow rate corresponded to a volume hourly space velocity (VHSV) of 10,150 h⁻¹ on the basis of the wet gas composition.

The subsequent activity test included heating the catalyst in the feed gas mixture at temperatures ramping up from 90° C. to 300° C. at a rate of 2° C./min.

The catalyst performance was characterized by constantly monitoring CO concentration at the reactor outlet with an on-line infrared gas analyzer ZRF (Fuji Electric, Japan). The CO outlet concentration hence is the measure of the catalytic activity: the lower the concentration, the higher the activity. Table 3 summarizes the CO outlet concentrations for the two catalysts at various temperatures between 190 and 290° C. As can be seen, the wood flour modified Catalyst A1 yielded less CO than Catalyst B1 which had exactly the same amounts of copper component and alumina, but which catalyst was not formed with wood flour.

TABLE 3

CO Outlet Concentration (%)

| Temperature (° C.) | Catalyst A1 | Catalyst B1 |
|---|---|---|
| 190 | 2.83 | 3.34 |
| 210 | 1.88 | 2.20 |
| 230 | 0.99 | 1.27 |
| 250 | 0.76 | 1.04 |
| 270 | 0.74 | 0.98 |
| 290 | 0.75 | 0.93 |

### EXAMPLE 4

### Evaluation of Aging Rate—Effect of Wood Flour Addition

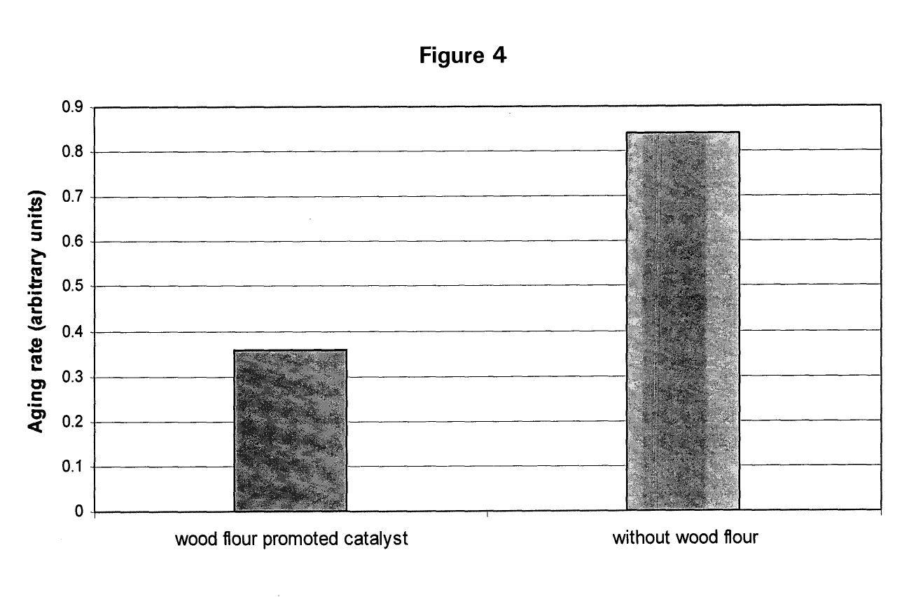

The reference catalyst, Catalyst B1 (prepared without the addition of wood flour), was aged according to the aging protocol described in Example 2. The comparative Aging Rates for Catalyst A1 (prepared with the addition of wood flour) and Catalyst B1 prepared are shown in FIG. 4. As can be seen in the figure, the aging rate for Catalyst B1 is greater than twice the aging rate for the Catalyst A1.

As the compositions for Catalysts A1 and B1 were otherwise identical, the addition of the carbon precursor (wood flour) to Catalyst A1 accounts for the increased longevity of its WGS activity in this experiment.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

1. A multi-cyclic method of operating a water-gas shift (WGS) reactor comprising a copper-based WGS catalyst, the method comprising:
(a) heating the WGS catalyst in its oxidized state in a substantially dry, reducing gas comprising a reducing component to a temperature of 100 to 350° C. to form a reduced WGS catalyst;
(b) flowing a reactant gas comprising CO and H₂O through the reactor to contact the reduced WGS catalyst, while maintaining the WGS catalyst temperature at an operating temperature of 150 to 350° C.;
(c) purging the WGS reactor with a substantially dry, first non-oxidizing gas at the operating temperature, and allowing the WGS catalyst temperature to fall below the operating temperature;
(d) heating the WGS catalyst to the operating temperature in a substantially dry, second non-oxidizing gas; and
(e) repeating (b) through (d) at least two times.

2. The multi-cyclic method of claim 1, wherein the reducing component in the reducing gas of (a) comprises at least 1% by volume of CO, H₂, or a mixture thereof.

3. The multi-cyclic method of claim 2, wherein the reducing gas of (a) is derived from an upstream reformer reactor operating in catalytic partial oxidation mode.

4. The multi-cyclic method of claim 1, wherein the substantially dry, first and second non-oxidizing gases consist essentially of at least one of nitrogen, argon, de-sulfured gaseous hydrocarbon, carbon dioxide, carbon monoxide and hydrogen.

5. The multi-cyclic method of claim 1, wherein the substantially dry, first non-oxidizing gas has the same composition as the substantially dry, second non-oxidizing gas.

6. The multi-cyclic method of claim 1, wherein the reactant gas of (b) further comprises at least 10% by volume H₂, on a dry basis.

7. The multi-cyclic method of claim 1, wherein the copper-based, WGS catalyst comprises from 5 to 20 wt. % of a copper component, wherein at least 50 wt. % of the copper component is in the form of a copper oxide, aluminum oxide spinel; and at least 75 wt. % of alumina.

8. The multi-cyclic method of claim 7, wherein in (a), the WGS catalyst in its oxidized state is heated in the reducing gas at a temperature below 300° C. to form the reduced WGS catalyst.

9. The multi-cyclic method of claim 7, wherein in (b), the reduced WGS catalyst is contacted with the reactant gas, while maintaining the WGS catalyst temperature at an operating temperature of 180 to 300° C.

10. A multi-cyclic method of operating a water-gas shift (WGS) reactor comprising a copper-based WGS catalyst, the method comprising:
(a) heating the WGS catalyst in its oxidized state in a reducing gas comprising a reducing component to a temperature of 150 to 350° C. to form a reduced WGS catalyst;
(b) flowing a reactant gas comprising CO and H₂O through the reactor to contact the reduced WGS catalyst, while maintaining the WGS catalyst temperature at an operating temperature of 180 to 300° C.;
(c) purging the WGS reactor with a substantially dry, first non-oxidizing gas at the operating temperature, and allowing the WGS catalyst temperature to fall below the operating temperature;
(d) heating the WGS catalyst to the operating temperature in a substantially dry, second non-oxidizing gas; and
(e) repeating (b) through (d) at least two times;
wherein the copper-based, WGS catalyst comprises from 5 to 20 wt. % of a copper component, wherein at least 50 wt. % of the copper component is in the form of a copper oxide, aluminum oxide spinel; and at least 75 wt. % of alumina.

11. The multi-cyclic method of claim 10, wherein the copper-based, WGS catalyst further comprises from 0.03 to 1 wt. % of carbonaceous residue.

12. A catalyst comprising:
from 5 to 20 wt. % of a copper component, wherein at least 50 wt. % of the copper component is in the form of a copper oxide, aluminum oxide spinel;
at least 75 wt. % of alumina; and
from 0.03 to 1 wt. % of carbonaceous residue.

13. A method of conducting the water-gas shift reaction comprising:
contacting a reactant gas stream comprising CO and H₂O with a WGS catalyst to form H₂ and CO₂;
wherein the WGS catalyst comprises:
from 5 to 20 wt. % of a copper component, wherein at least 50 wt. % of the copper component is in the form of a copper oxide, aluminum oxide spinel;
at least 75 wt. % of alumina; and
from 0.03 to 1 wt. % of carbonaceous residue.
